(19) United States
(12) Patent Application Publication
(10) Pub. No.: US 2023/0061631 A1
(43) Pub. Date: Mar. 2, 2023

(54) ENDOSCOPE FORCEPS VALVE COVER

(71) Applicant: FUJIFILM Corporation, Tokyo (JP)

(72) Inventors: Takumi DEJIMA, Kanagawa (JP); Tomohiro OHKI, Kanagawa (JP); Ryo ISHIKAWA, Kanagawa (JP)

(73) Assignee: FUJIFILM Corporation, Tokyo (JP)

(21) Appl. No.: 18/053,002

(22) Filed: Nov. 7, 2022

Related U.S. Application Data
(63) Continuation of application No. PCT/JP2021/017377, filed on May 6, 2021.

(30) Foreign Application Priority Data
May 8, 2020 (JP) 2020-082834

(51) Int. Cl. A61B 17/29; A61B 1/00; A61B 1/018

(52) U.S. Cl. CPC A61B 17/29; A61B 1/00137; A61B 1/018

(57) ABSTRACT

A forceps valve is provided at a forceps port of the endoscope. An endoscope forceps valve cover includes a cover body, a drape, and a fluid suppression member. The cover body is mounted on the forceps valve. The drape is provided integrally with the cover body and covers at least a part of a user who operates the endoscope. The fluid suppression member suppresses leakage of a fluid from the forceps valve to an outside of the endoscope in a case where the cover body is mounted on the forceps valve.

### CROSS-REFERENCE TO RELATED APPLICATIONS

This application is a Continuation of PCT International Application No. PCT/JP2021/017377 filed on 6 May 2021, which claims priority under 35 U.S.C. § 119(a) to Japanese Patent Application No. 2020-082834 filed on 8 May 2020. The above application is hereby expressly incorporated by reference, in its entirety, into the present application.

### BACKGROUND OF THE INVENTION

### 1. Field of the Invention

The present invention relates to an endoscope forceps valve cover that is used by being mounted on a forceps valve of an endoscope.

### 2. Description of the Related Art

In the medical field, insertion parts of endoscopes are inserted into a body of a subject to perform not only observation inside the body but also various treatments on observation sites. Specifically, various treatments such as excision and collection of the observation sites are performed by inserting various treatment tools such as forceps and incision tools through a forceps channel in an insertion part from a forceps port provided in an operating part of an endoscope and delivering the treatment tools from a forceps outlet opening to a distal end of the insertion part.

The forceps port of the endoscope is provided with a forceps valve through which a treatment tool can be inserted. The forceps valve is provided with, for example, a forceps insertion hole having a hole diameter slightly smaller than that of the forceps and a slit valve formed with a slit through which the forceps are inserted. In a state in which the treatment tool is inserted through the forceps valve, there is a possibility that the slit valve is spread and a gap is created between the slit valve and an outer peripheral surface of the treatment tool at a portion on an end of the slit, and contents such as a body fluid such as blood and residues in the body leak from the portion.

Thus, it is known to use a contamination liquid scattering preventive tool to be mounted on the forceps valve of the endoscope (see JP1998-57302A (JP-H10-57302A) and JP1998-99258A (JP-H10-99258A, corresponding to U.S. Pat. No. 5,993,379A). The contamination liquid scattering preventive tool is provided with a contamination liquid absorbing member made of a water-absorbing material. In the contamination liquid scattering preventive tool described in JP1998-57302A (JP-H10-57302A), the contamination liquid absorbing member is disposed to face an inlet of the forceps valve. The contamination liquid absorbing member has a dividing surface through which the treatment tool is passed. In a case where the contents such as body fluids leak from the forceps valve, the contamination liquid absorbing member absorbs the contents.

Meanwhile, the contamination liquid scattering preventive tool described in JP1998-99258A (JP-H10-99258A) is formed with a mounting hole into which a mouthpiece that forms the forceps port is inserted. The mouthpiece is inserted into the mounting hole of the contamination liquid scattering preventive tool, and the forceps valve is attached to a distal end side of the mouthpiece from above to retain the contamination liquid scattering preventive tool. The contamination liquid scattering preventive tool attached to the mouthpiece is folded back in a U shape and covers an inlet portion of the forceps valve.

### SUMMARY OF THE INVENTION

In the medical field, it is desired to take further preventive measures against infectious diseases. In particular, it is important to take measures to prevent infection caused by contact with the contents such as body fluids of infectious disease patients with mucous membranes of eyes or mouths.

However, in the contamination liquid scattering preventive tools described in JP1998-57302A (JP-H10-57302A) and JP1998-99258A (JP-H10-99258A), a case where the treatment tool is removed from a state in which the treatment tool is inserted through the forceps valve, or a case where the pressure in a body cavity increases and the contents are spouted is not taken into account. In a case where the treatment tool is removed from the forceps valve, there is a possibility that the contents are scattered by the momentum of the removed treatment tool. In the contamination liquid scattering preventive tool described in JP1998-57302A (JP-H10-57302A), in a case where the contents are scattered, there is a possibility that the contents leak from the dividing surface of the contamination liquid absorbing member.

Meanwhile, in the contamination liquid scattering preventive tool described in JP1998-99258A (JP-H10-99258A), there is a space between the contamination liquid scattering preventive tool folded back in a U shape and the forceps valve, and in a case where the contents are spouted, there is a possibility that the contents are scattered into the space between the forceps valve and the contamination liquid scattering preventive tool. In a case where the contents are scattered or spouted, there is a possibility that the scattered or spouted contents come into contact with the mucous membranes of persons around the patient, such as an operator or a helper.

An object of the present invention is to provide an endoscope forceps valve cover capable of preventing diffusion of contents scattered or spouted from a forceps valve by the momentum of a removed treatment tool.

An endoscope forceps valve cover according to an aspect of the present invention includes a cover body, a drape, and a fluid suppression member. The cover body is mounted on a forceps valve provided at a forceps port of an endoscope into which a treatment tool is inserted, and has a through-hole through which the treatment tool is inserted. The drape is provided integrally with the cover body, is located on a side facing an outside of the endoscope with respect to the cover body, and covers at least a part of a user who operates the endoscope. The fluid suppression member is provided integrally with the cover body and the drape, is located on the side facing the outside of the endoscope with respect to the drape, and is disposed at a position facing the forceps port to suppress leakage of a fluid from the forceps valve to the outside of the endoscope in a case where the cover body is mounted on the forceps valve.

It is preferable that the cover body includes a plurality of protruding portions that are formed of an elastic member and that protrude toward the endoscope in a case where the through-hole is aligned with a position of the forceps port, and is mounted on the forceps valve by an elastic force of the protruding portion.

It is preferable that the cover body includes a protruding portion that is formed of an elastic member, protrudes toward the endoscope, and is formed in a tubular shape having a notched portion in a case where the through-hole is aligned with a position of the forceps port, and is mounted on the forceps valve by an elastic force of the protruding portion.

It is preferable that the fluid suppression member is formed of a flexible porous member, has a slit through which the treatment tool is inserted, and is compressed in an axial direction in a case where the treatment tool is inserted through the slit. It is preferable that the fluid suppression member has a columnar shape having an axial dimension longer than a radial dimension. It is more preferable that the fluid suppression member has an axial dimension of 10 mm or more and 20 mm or less.

It is preferable that the slit is a first slit parallel to an insertion direction of the treatment tool and a second slit intersecting the first slit.

According to the present invention, the diffusion of contents scattered or spouted from the forceps valve can be prevented by the momentum of the removed treatment tool.

### BRIEF DESCRIPTION OF THE DRAWINGS

FIG. 1 is a schematic view showing endoscopy using an endoscope and a treatment tool.

FIG. 2 is a perspective view of the endoscope, the treatment tool, and an endoscope forceps valve cover.

FIG. 3 is a perspective view of the endoscope forceps valve cover and the treatment tool in a used state.

FIG. 4 is a perspective view of the endoscope forceps valve cover in an unused state.

FIG. 5 is a perspective view of the endoscope forceps valve cover and a forceps valve.

FIG. 6 is an exploded perspective view showing a configuration of the endoscope forceps valve cover.

FIG. 7 is a cross-sectional view showing the configuration of the endoscope forceps valve cover.

FIG. 8 is a cross-sectional view showing a state in which the treatment tool is inserted into the forceps valve through the endoscope forceps valve cover.

FIG. 9 is an explanatory view illustrating a state in which the treatment tool is removed through a fluid suppression member.

FIGS. 10A and 10B are explanatory views illustrating a method of folding a drape in a front-back direction.

FIGS. 11A and 11B are explanatory views illustrating a method of folding the drape in a longitudinal direction.

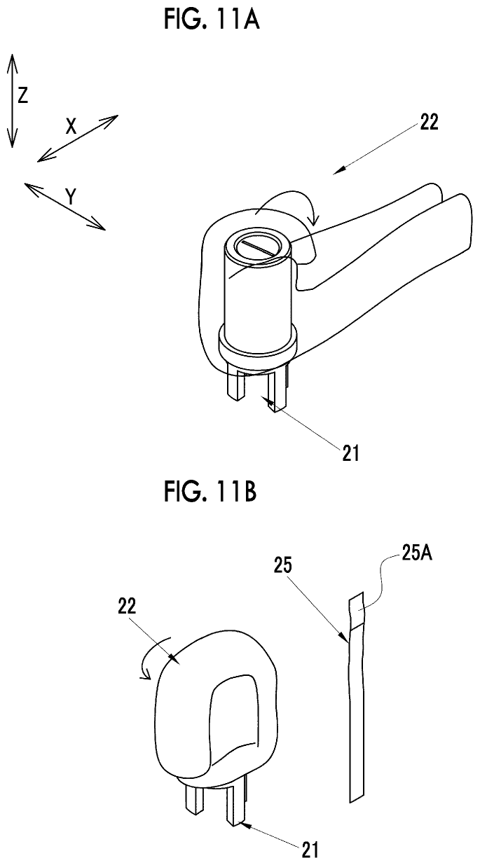

### DESCRIPTION OF THE PREFERRED EMBODIMENTS

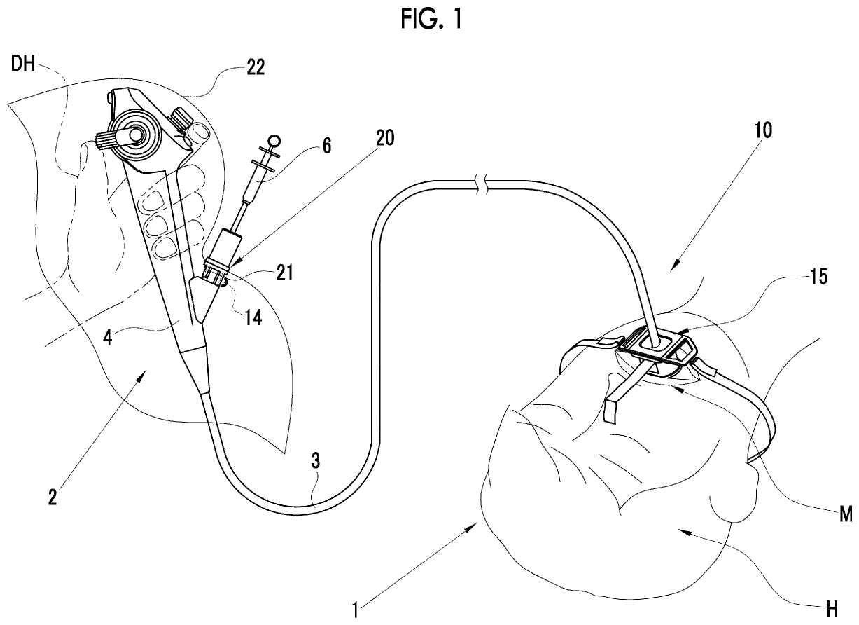

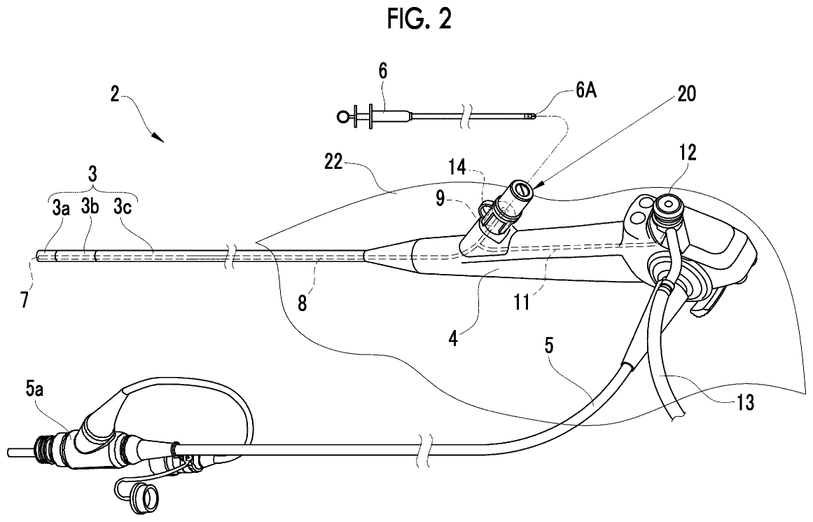

As shown in FIG. 1, an endoscope forceps valve cover 20 of the embodiment of the present invention is used for endoscopy using an endoscope 2. The endoscope 2 is, for example, a bronchoscope to be inserted into the trachea, and comprises an insertion part 3 inserted into a trachea of a patient 1 who is a subject, an operating part 4 consecutively installed at a proximal end part of the insertion part 3, and a universal cord 5 (see FIG. 2) connected to the operating part 4. The universal cord 5 is connected to an external device such as a processor device or a light source device (not shown) via a composite type connector 5a (see FIG. 2).

As shown in FIG. 2, the insertion part 3 includes a distal rigid part 3a, a bendable part 3b, and a flexible tube part 3c having flexibility, in this order toward a proximal end side from a distal end side. An observation window and an illumination window in addition to a forceps outlet 7, which is an outlet of a treatment tool 6 such as forceps, are provided (not shown) on a distal end surface of the distal rigid part 3a. An image sensor (not shown) is disposed behind the observation window, and an optical fiber cable (not shown) is disposed behind the illumination window. A signal line of the image sensor and the optical fiber cable are connected to a processor device and a light source device, respectively, through the insertion part 3, the operating part 4, the universal cord 5, and the connector 5a. The bendable part 3b is consecutively installed at the distal rigid part 3a and is provided to be bendable.

In a case where the insertion part 3 of the endoscope 2 is inserted from a mouth M of the patient 1, an endoscope mouthpiece 15 is mounted on the mouth of the patient 1. The endoscope mouthpiece 15 has a pipe line (not shown) that allows the insertion part 3 of the endoscope to be inserted therethrough. The endoscope mouthpiece 15 is mounted on the mouth M of the patient 1 by inserting a part thereof into the mouth of the patient 1 and by the patient 1 holding the inserted portion by the mouth. Accordingly, it is possible to introduce the insertion part 3 into a body through the pipe line, and the insertion part 3 can be prevented from being damaged by teeth of the patient 1.

A forceps channel 8 for allowing the treatment tool 6 to be inserted therethrough is disposed in the insertion part 3. One end of the forceps channel 8 is connected to the forceps outlet 7, and the other end thereof is connected to a forceps port 9 provided in the operating part 4. Additionally, the forceps channel 8 is also used as a route for suctioning a body fluid such as blood and contents such as body waste from the forceps outlet 7. A suction channel 11 branched from the forceps channel 8 is disposed in the operating part 4, and the suction channel 11 is connected to a suction button 12 provided in the operating part 4.

The suction button 12 includes a suction valve (not shown) provided inside. The suction valve is connected to the suction channel 11 in the operating part 4 and is connected to an external suction pump (not shown) via a suction tube 13 connected to the suction button 12. By operating to press a pressing unit of the suction button 12, a shaft portion of the suction valve slides, and the suction channel 11 and a pipe line of the suction pump communicate with each other. Accordingly, the body fluid or the like can be suctioned from the forceps outlet 7 of the insertion part 3 inserted into the subject or the like. Additionally, by releasing the pressing operation of the pressing unit, the communication between the suction channel 11 and the pipe line of the suction pump is cut off, and the suction from the forceps outlet 7 can be stopped.

The forceps port 9 is provided with a forceps valve 14. The forceps valve 14 is mounted with an endoscope forceps valve cover 20.

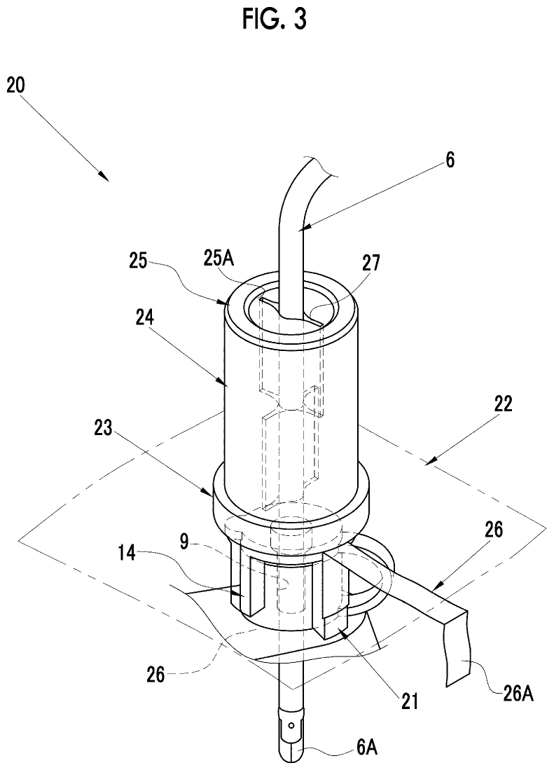

As shown in FIG. 3, the endoscope forceps valve cover 20 comprises a cover body 21, a drape 22, a retaining member 23, a fluid suppression member 24, a lid member 25, and a pressure-sensitive adhesive tape 26. The drape 22 has a small thickness and is formed in the shape of a quadrangular sheet, for example, is formed of a transparent vinyl sheet. The drape 22 has an area that covers and hides at least a part of a doctor who is a user who operates the endoscope 2, for example, a hand DH (see FIG. 1) of the doctor.

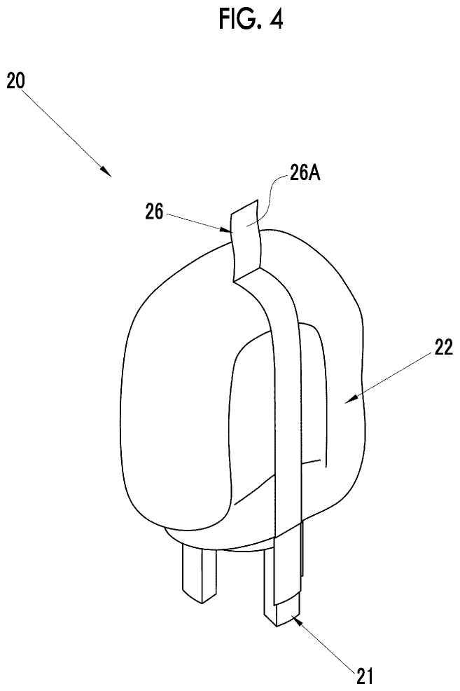

As shown in FIG. 4, the drape 22 is folded in the case of an unused state and has the pressure-sensitive adhesive tape 26 as a holding member stuck on an end part thereof. By applying the pressure-sensitive adhesive tape 26, the drape 22 is held in the folded state. In addition, the method of folding the drape 22 and the method of applying the pressure-sensitive adhesive tape 26 will be described below.

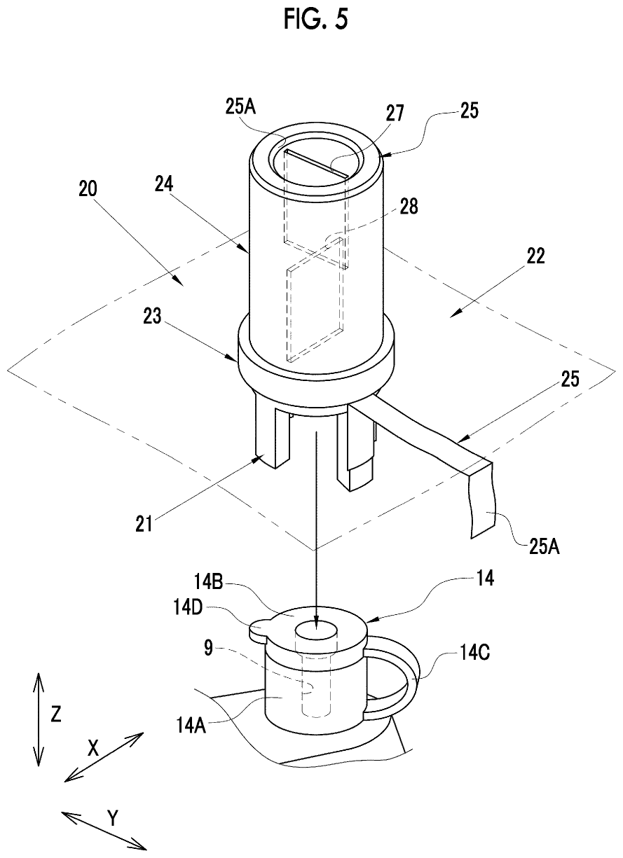

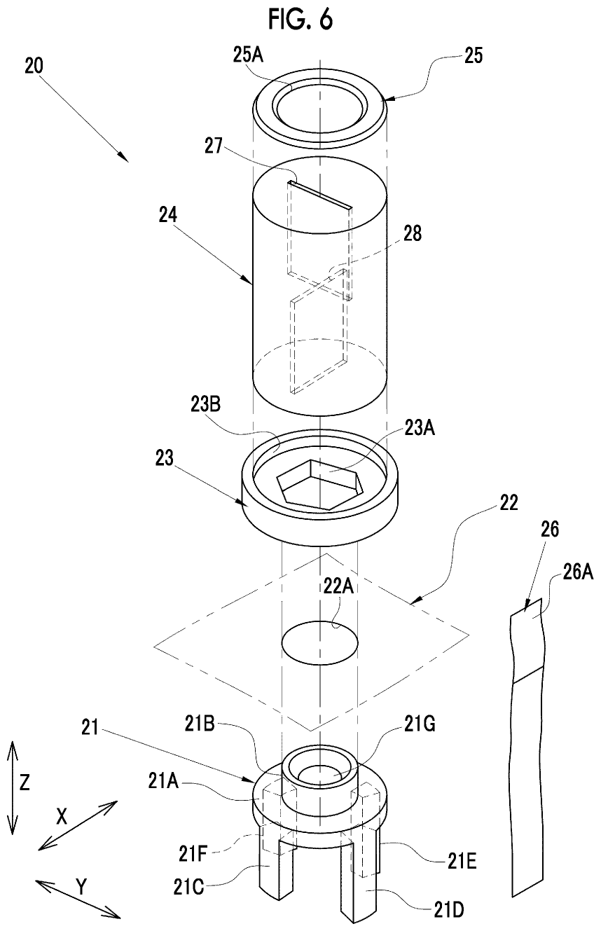

As shown in FIG. 5, the drape 22 is formed with an opening portion 22A that matches the outer shape of a fitting portion 21B (described below) of the cover body 21. By aligning the opening portion 22A with the position of the fitting portion 21B, the drape 22 can be positioned with respect to the cover body 21. In addition, for convenience of illustration, the drape 22 in FIGS. 5 and 6 is shown by cutting out only the periphery of the opening portion 22A (a range surrounded by a two-dot chain line), and the actual size thereof is larger than the range surrounded by the two-dot chain line.

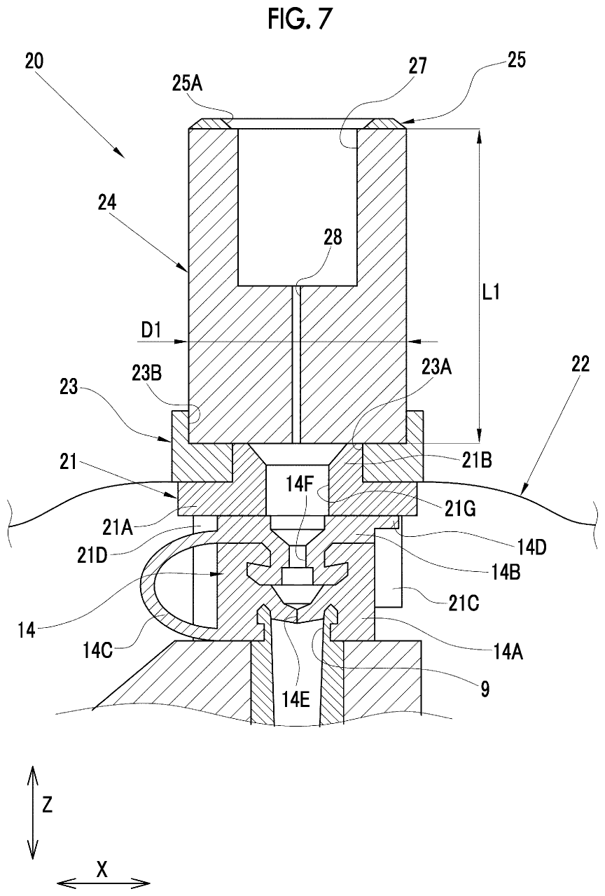

The forceps valve 14 includes, for example, a cylindrical valve body 14A, a cap 14B that fits to the valve body 14A, and an attachment arm 14C. Additionally, the cap 14B has a knob portion 14D that protrudes from the outer diameter. The attachment arm 14C connects the valve body 14A and the cap 14B to each other. A slit valve 14E (see FIG. 7) formed with a slit through which the treatment tool 6 is inserted is provided inside the valve body 14A. The cap 14B is provided with a forceps insertion hole 14F (see FIG. 7) having a hole diameter slightly smaller than the outer diameter of the treatment tool 6.

As shown in FIG. 6, the cover body 21 is integrally formed with a disk portion 21A, a fitting portion 21B, a plurality of protruding portions 21C to 21F, and a through-hole 21G. The disk portion 21A has a larger outer diameter than the forceps valve 14. The fitting portion 21B has a cylindrical shape that is coaxial with the disk portion 21A and has an outer diameter smaller than that of the disk portion 21A. The plurality of protruding portions 21C to 21F are continuous with an outer peripheral surface of the disk portion 21A and protrude from a surface opposite to the fitting portion 21B. The cover body 21 is formed of an elastic member, for example, rubber. The through-hole 21G is formed in an axial direction of the disk portion 21A and the fitting portion 21B.

The protruding portions 21C to 21F extend in a direction in which the protruding portions protrude toward the endoscope 2 in a case where the through-hole 21G is aligned with the position of the forceps port 9. The protruding portions 21C to 21F are formed at positions and sizes that do not interfere with the attachment arm 14C and the knob portion 14D of the forceps valve 14. The protruding portions 21C to 21F are formed such that the curvature radius of an inner peripheral surface of each protruding portion is slightly smaller than the outer diameter of the forceps valve 14. Due to the elastic force of the protruding portions 21C to 21F, the protruding portions 21C to 21F come into close contact with an outer peripheral surface of the forceps valve 14. Accordingly, the endoscope forceps valve cover 20 is mounted on the forceps valve 14.

The retaining member 23 is formed in a disk shape having the same or a slightly larger outer diameter than the cover body 21. The retaining member 23 is formed of, for example, a soft material such as a soft resin. The retaining member 23 is formed with a fitting hole 23A and an opening portion 23B. The fitting hole 23A is a hexagonal through-hole located at the center of the retaining member 23. The diameter of an inscribed circle of the fitting hole 23A is formed to be slightly smaller than the outer diameter of the fitting portion 21B of the cover body 21. By fitting the fitting hole 23A with the fitting portion 21B, the retaining member 23 is combined with the cover body 21.

As shown in FIG. 6, the retaining member 23 sandwiches the drape 22 with the cover body 21. In this case, the retaining member 23 can be anchored to the cover body 21 by fitting the fitting portion 21B of the cover body 21 into the opening portion 22A of the drape 22 and the fitting hole 23A of the retaining member 23.

The drape 22 is provided integrally with the cover body 21 by being sandwiched between the retaining member 23 and the cover body 21. Accordingly, in a case where the drape 22 is mounted on the forceps valve 14 together with the cover body 21 and the retaining member 23, the drape 22 is located on a side facing an outside of the endoscope 2 with respect to the cover body 21.

The opening portion 23B is provided at an end on the side located on the outside of the endoscope 2 in a case where the retaining member 23 is mounted on the forceps valve 14 together with the cover body 21. The fluid suppression member 24 is anchored to the opening portion 23B. Accordingly, the fluid suppression member 24 is provided integrally with the cover body 21 and the drape 22.

The fluid suppression member 24 is disposed at a position facing the forceps port 9 to prevent fluid from leaking from the forceps valve 14 to the outside of the endoscope 2. Specifically, the fluid suppression member 24 allows a gas such as air to pass therethrough and blocks liquids and solids containing contents such as body fluids. In a case where the cover body 21 is mounted on the forceps valve 14, the fluid suppression member 24 faces the forceps port 9 and is disposed outside the endoscope 2.

The lid member 25 is formed in a disk shape having an outer diameter matching that of the fluid suppression member 24, and is formed of, for example, resin. The lid member 25 has a through-hole 25A. The through-hole 25A exposes a slit 27, which will be described below, of the fluid suppression member 24.

The fluid suppression member 24 is formed by forming a flexible porous material into a columnar shape. The fluid suppression member 24 has an axial dimension L1 longer than a radial dimension D1. The axial dimension L1 is preferably, for example, 10 mm or more and 20 mm or less. Accordingly, the fluid suppression member 24 can sufficiently suppress the passage of the fluid, and allows the treatment tool 6 to be smoothly inserted thereinto.

The fluid suppression member 24 has a first slit 27 through which the treatment tool 6 is inserted, and a second slit 28. The first slit 27 is formed parallel to an insertion direction Z of the treatment tool 6 and parallel to a front-back direction Y. The front-back direction Y is a direction perpendicular to the insertion direction Z.

The second slit 28 is formed in a direction parallel to the insertion direction Z and intersecting the first slit 27. More specifically, the second slit 28 is formed parallel to the insertion direction Z and parallel to a left-right direction X. The left-right direction X is a direction perpendicular to the front-back direction Y and to the insertion direction Z. The position where the first slit 27 and the second slit 28 intersect each other is preferably the center of the fluid suppression member 24 in the axial direction and the radial direction.

The porous material that forms the fluid suppression member 24 is a porous material having a pore diameter and a structure that allows a gas such as air to pass therethrough and that blocks liquids and solids containing contents such as body fluids. For example, a synthetic sponge obtained by foam-molding a resin or a natural sponge such as a sponge is used.

As shown in FIG. 7, one end part of the fluid suppression member 24 is anchored to the opening portion 23B, and the lid member 25 is anchored to the other end part. In this case, for example, the retaining member 23 and the fluid suppression member 24 are anchored to each other by adhesion or pressure-bonding, and the fluid suppression member 24 and the lid member 25 are anchored to each other.

By anchoring the fluid suppression member 24 to the retaining member 23 as described above, the fluid suppression member 24 is located on the side facing the outside of the endoscope 2 with respect to the drape 22. Accordingly, the fluid suppression member 24 is disposed at a position facing the forceps port 9 in a case where the cover body 21 is mounted on the forceps valve 14.

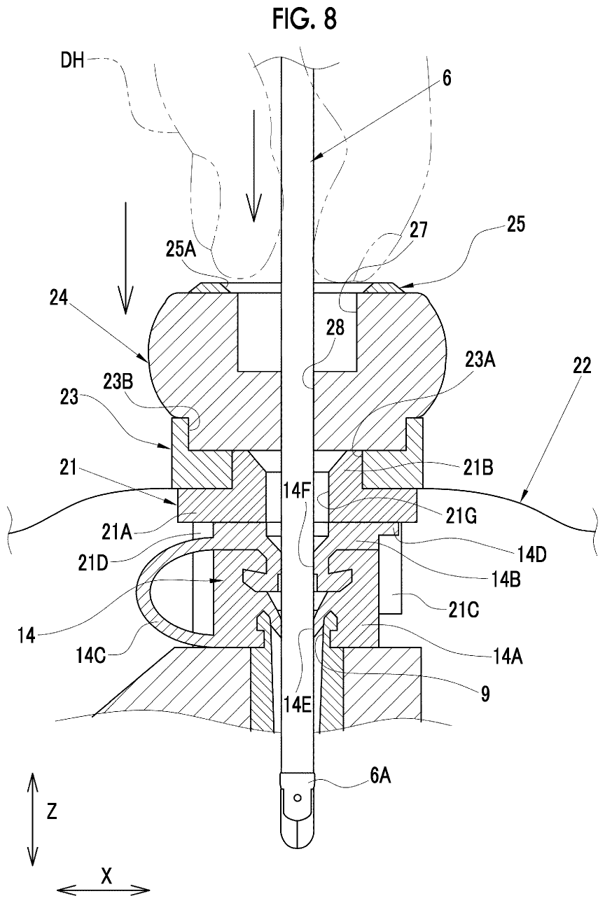

As shown in FIG. 8, in a case where the treatment tool 6 is inserted into the forceps channel 8 through the fluid suppression member 24, the insertion part 3 moves in the insertion direction Z while can outer peripheral surface of the insertion part 3 comes into close contact with the first slit 27 and the second slit 28. Since the fluid suppression member 24 has flexibility as described above, in a case where the treatment tool 6 is inserted into the first slit 27 and the second slit 28, the fluid suppression member 24 receives pressure from the treatment tool 6 or the hand DH of the doctor and is compressed in the axial direction. The hand DH of the doctor that grips and pushes the treatment tool 6 is brought to a position closer to the forceps valve 14 by the amount by which the fluid suppression member 24 is compressed. If the treatment tool 6 is gripped far away from the forceps valve 14 and is then pushed in, the treatment tool 6 buckles. However, in the endoscope forceps valve cover 20 of the present embodiment, the treatment tool 6 is gripped at a position close to the forceps valve 14. Therefore, the buckling of the treatment tool 6 can be prevented.

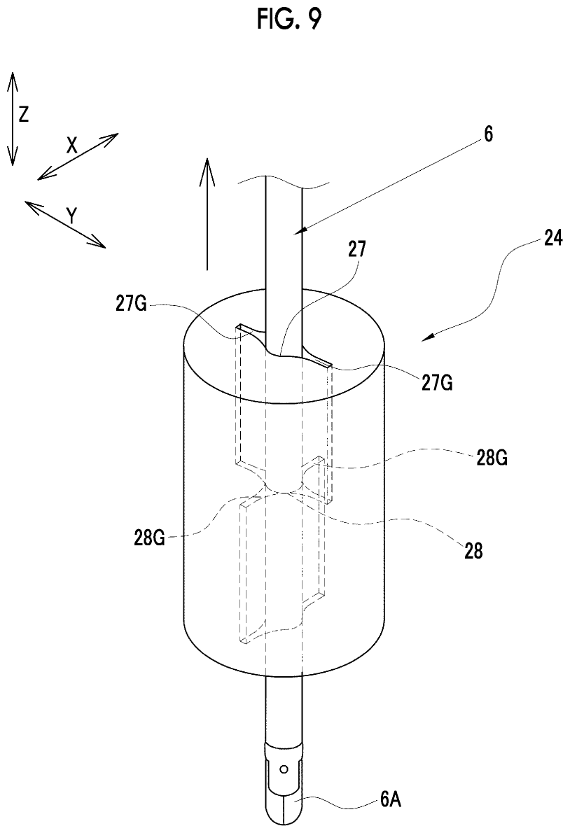

As shown in FIG. 9, in a case where the treatment tool 6 is inserted into the forceps channel 8, gaps 27G and 28G are formed at both end parts of the first slit 27 and the second slit 28. However, since the first slit 27 and the second slit 28 intersect each other as described above, the positions of the gaps 27G and 28G do not overlap each other. In addition, since FIG. 9 shows the fluid suppression member 24 in a case where the treatment tool 6 is removed from the forceps valve 14, the fluid suppression member 24 has returned to the original dimensions thereof from the compressed state.

As described above, the drape 22 sandwiched between the cover body 21 and the lid member 25 protrudes from an outer peripheral edge of the cover body 21 and is provided integrally with the cover body 21 (a state shown in FIG. 3). On the other hand, the drape 22 is in the folded state when the endoscope forceps valve cover 20 is not in use, and the folded state is maintained by the pressure-sensitive adhesive tape 26 (a state shown in FIG. 4). A method of folding the drape 22 and a method of applying the pressure-sensitive adhesive tape 26 will be described below.

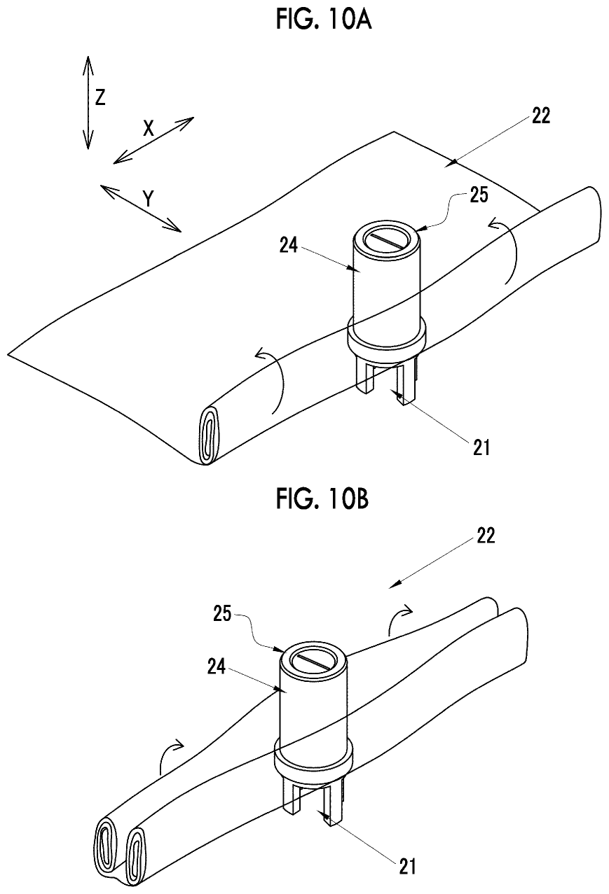

As shown in FIG. 10A, in a case where the drape 22 is folded, for example, the drape 22 is rolled from one end in the front-back direction Y, and the drape 22 is rolled to the position of the fluid suppression member 24 located at the center, and then, the drape 22 is also rolled from the other end in the front-back direction Y. Accordingly, as shown in FIG. 10B, the drape 22 has an elongated belt shape.

Next, as shown in FIG. 10A, the drape 22 is folded to be rolled from one end having the elongated belt shape. Then, after the drape 22 is rolled to the position of the fluid suppression member 24 located at the center, the drape 22 is also rolled from the other end having the elongated belt shape. In this case, the fluid suppression member 24 is simultaneously covered and hidden with the drape 22. Accordingly, as shown in FIG. 10B, the drape 22 is in a small folded state. Then, the pressure-sensitive adhesive tape 26 is stuck on an end part of the drape 22 from the cover body 21. An end part 26A of the pressure-sensitive adhesive tape 26 does not adhere to the drape 22 but is made to protrude from the outer shape of the drape 22. In this case, for example, the end part 26A is folded back, and the pressure-sensitive adhesive surfaces thereof are attached to each other in advance to form a portion where the pressure-sensitive adhesive surfaces are not exposed, on the pressure-sensitive adhesive tape 26.

By applying the pressure-sensitive adhesive tape 26 as described above, the folded state of the drape 22 is maintained (the state shown in FIG. 4). Additionally, by making the end part 26A not adhere to the drape 22, in a case where the endoscope forceps valve cover 20 is used, it is easy to grip the end part 26A and to peel off the pressure-sensitive adhesive tape 26.

The operation in a case where the doctor, who is the user, mounts the endoscope forceps valve cover 20 on the forceps valve 14 of the endoscope 2 and inserts the treatment tool 6 into the forceps channel 8 through the forceps port 9 will be described. As shown in FIG. 1, first, the doctor mounts the endoscope forceps valve cover 20 on the forceps valve 14 by means of the elastic force of the protruding portions 21C to 21F of the cover body 21.

By peeling off the pressure-sensitive adhesive tape 26 after the endoscope forceps valve cover 20 is mounted on the forceps valve 14, the drape 22 can be brought to an original state, that is, a spread state thereof from the folded state. Then, by mounting the endoscope forceps valve cover 20 on the forceps valve 14 and bringing the drape 22 into a spread state, the hand DH of the doctor who operates the endoscope 2, the operating part 4, and the like are covered and hidden with the drape 22.

Meanwhile, the doctor makes the endoscope mouthpiece 15 to be worn on the mouth M. After the endoscope mouthpiece 15 is worn on the mouth M, the doctor inserts the insertion part 3 of the endoscope 2 into the body of the patient 1 through the endoscope mouthpiece 15 from the mouth M. Moreover, the treatment tool 6 is inserted into the forceps channel 8 through the slits 27 and 28 of the endoscope forceps valve cover 20, the slit valve 14E and the forceps insertion hole 14F of the forceps valve 14, and the forceps port 9. By making a distal end part 6A of the treatment tool 6 protrude from the forceps outlet 7 of the distal rigid part 3a, the doctor can perform various treatments such as excision and collection of an observation site.

By performing various treatments, contents such as the body fluid of the patient 1 adhere to the treatment tool 6. In a case where the treatment tool 6 is removed from the forceps valve 14, the doctor removes the treatment tool 6 from the forceps valve 14 together with the endoscope forceps valve cover 20 while wrapping the treatment tool 6 with the drape 22. In addition, the endoscope forceps valve cover 20 is preferably a disposable type that is removed from the forceps valve 14 and then discarded together with the drape 22.

Even in a case where the contents are scattered from the forceps valve 14 by the momentum of the removed treatment tool 6, the fluid suppression member 24 absorbs the contents and blocks passing of the contents to the outside of the endoscope 2. Additionally, even in a case where the contents are spouted from the forceps valve 14, the contents are wrapped in the drape 22 such that the contents can be prevented from being diffused. Thus, since the body fluid discharged from the body of the patient 1 can be prevented from coming into contact with the mucous membrane of a person around the patient such as an operator or a helper, infection can be reliably prevented.

Additionally, the treatment tool 6 is inserted into the forceps valve 14 through the fluid suppression member 24. However, since the fluid suppression member 24 is formed to have a long axial dimension, the passage of the contents can be sufficiently blocked. Additionally, since the first slit 27 and the second slit 28 intersect as described above, the positions of the gaps 27G and 28G do not overlap each other. That is, the body fluid can be prevented from leaking from the fluid suppression member 24 even in a state in which the treatment tool 6 is inserted or not inserted through the fluid suppression member 24, or even while the treatment tool 6 is being removed.

In addition, in the above embodiment, the position of the fluid suppression member 24 with respect to the drape 22 is restricted by being anchored to the retaining member 14. However, the present invention is not limited to this, and the fluid suppression member 24 may be fixed to the drape 22 by adhesion or the like.

Additionally, in the above embodiment, the cover body 21 has the four protruding portions 21C to 21F for mounting on the forceps valve 14. However, the cover body 21 is not limited to this, and only needs to have a plurality of protruding portions as in the above embodiment. Alternatively, the protruding portions for mounting on the forceps valve 14 may include one protruding portion. For example, in a case where the through-hole 21G of the cover body 21 is aligned with the position of the forceps port 9, a protruding portion that protrudes toward the endoscope 2 and that is formed a tubular shape having a notched portion may be used. In this case, the notched portion of the protruding portion is formed to match the position and size of the attachment arm 14C and the knob portion 14D of the forceps valve 14. By configuring the protruding portion as described above, an inner peripheral surface of the protruding portion comes into close contact with the outer peripheral surface of the forceps valve 14. Accordingly, the endoscope forceps valve cover is mounted on the forceps valve 14.

In each of the above embodiments, the pressure-sensitive adhesive tape 26 is exemplified as the holding member for holding the drape 22 in the folded state. However, the present invention is not limited to this, and the holding member may include any one of a locking member, a constraint member, and a coating member. It is preferable that the locking member as the holding member has a configuration in which at least a part of each of the drapes 22 is locked, for example, by a locking structure consisting of a locking claw and a locking hole, a button and a button hole used for clothes, a fitting structure in which a male button and a female button are fitted to each other, or the like. Additionally, as the constraint member as the holding member, a paper tape or a rubber band that constrains the periphery of each of the drapes 22 in the folded state is preferable. Additionally, it is preferable that the coating member as the holding member is a bag-shaped member that covers the entire drapes 22 in the folded state.

Additionally, in each of the above embodiments, examples in which the drapes 22 are formed from vinyl sheets are given. However, the present invention is not limited to this, and any sheet-like member that covers and hides at least a part of the subject may be adopted. For example, the drapes 22 may be formed from paper, cloth, or the like. Additionally, in each of the above embodiments, a bronchoscope is used as an example of the endoscope. However, the present invention is not limited to this, and any endoscope including a forceps valve may be used, and, for example, an upper gastrointestinal endoscope or a lower gastrointestinal endoscope may be adopted.

### EXPLANATION OF REFERENCES

1: patient
2: endoscope
3: insertion part
3a: distal rigid part
3b: bendable part
3c: flexible tube part
4: operating part
5: universal cord
5a: connector
6: treatment tool
6A: distal end part
7: forceps outlet
8: forceps channel
9: forceps port
11: suction channel
12: suction button
13: suction tube
14: forceps valve
14A: valve body
14B: cap
14C: attachment arm
14D: knob portion
14E: slit valve
14F: forceps insertion hole
15: endoscope mouthpiece
20: endoscope forceps valve cover
21: cover body
21A: disk portion
21B: fitting portion
21C, 21D, 21E, 21F: protruding portion
21G: through-hole
22: drape
22A: opening portion
23: retaining member
23A: fitting hole
23B: opening portion
24: fluid suppression member
25: lid member
25A: through-hole
26: pressure-sensitive adhesive tape
26A: end part
27: first slit
28: second slit
27G: gap
28G: gap
D1: radial dimension
L1: axial dimension
DH: doctor's hand
H: head
M: mouth
X: left-right direction
Y: front-back direction
Z: insertion direction

What is claimed is:

1. An endoscope forceps valve cover comprising:
a cover body that is mounted on a forceps valve provided at a forceps port of an endoscope into which a treatment tool is inserted, and has a through-hole through which the treatment tool is inserted;
a drape that is provided integrally with the cover body, is located on a side facing an outside of the endoscope with respect to the cover body, and covers at least a part of a user who operates the endoscope; and
a fluid suppression member that is provided integrally with the cover body and the drape, is located on the side facing the outside of the endoscope with respect to the drape, and is disposed at a position facing the forceps port to suppress leakage of a fluid from the forceps valve to the outside of the endoscope in a case where the cover body is mounted on the forceps valve.

2. The endoscope forceps valve cover according to claim 1,
wherein the cover body includes a plurality of protruding portions that are formed of an elastic member and that protrude toward the endoscope in a case where the through-hole is aligned with a position of the forceps port, and is mounted on the forceps valve by an elastic force of the protruding portion.

3. The endoscope forceps valve cover according to claim 1,
wherein the cover body includes a protruding portion that is formed of an elastic member, protrudes toward the endoscope in a case where the through-hole is aligned with a position of the forceps port, and is formed in a tubular shape having a notched portion, and is mounted on the forceps valve by an elastic force of the protruding portion.

4. The endoscope forceps valve cover according to claim 1,
wherein the fluid suppression member is formed of a flexible porous member, has a slit through which the treatment tool is inserted, and is compressed in an axial direction in a case where the treatment tool is inserted through the slit.

5. The endoscope forceps valve cover according to claim 4,
wherein the fluid suppression member has a columnar shape having an axial dimension longer than a radial dimension.

6. The endoscope forceps valve cover according to claim 5,
wherein the fluid suppression member has an axial dimension of 10 mm or more and 20 mm or less.

7. The endoscope forceps valve cover according to claim 4,
wherein the slit is a first slit parallel to an insertion direction of the treatment tool, and a second slit intersecting the first slit.
